(19) United States
(12) Patent Application Publication
(10) Pub. No.: US 2012/0302947 A1
(43) Pub. Date: Nov. 29, 2012

(54) ELECTRICAL NEEDLE-FREE INJECTOR SYSTEM

(75) Inventors: Nicky CANTON, Shenzhen (CN); Bart HEGEMAN, Alkmaar (NL)

(73) Assignee: EUROPEAN PHARMA GROUP LIMITED, Kowloon (HK)

(21) Appl. No.: 13/115,094

(22) Filed: May 24, 2011

(51) Int. Cl. A61M 5/30

(52) U.S. Cl. 604/68

(57) ABSTRACT

In one aspect of the invention, a system comprising a needle-free injector is disclosed. The system comprises a needle-free injector comprising a trigger assembly adapted to exert an injection force, the trigger assembly comprising a spring storing the injection force. The system also comprises a winding mechanism for priming the spring for the storing of the injection force, wherein the winding mechanism is electrically powered. According to another aspect of the present invention, an alternate needle-free injector system is disclosed. The system comprises a needle-free injector comprising a nozzle adapted to be loaded with an injectable. The system also comprises a dosing mechanism for loading the injectable into the nozzle, wherein the dosing mechanism is electrically powered.

### FIELD OF INVENTION

This invention relates to a needle-free injector system, and in particular an electric-powered rechargeable needle-free injector system.

### BACKGROUND OF INVENTION

Conventional needle-free injectors are manually prepared for injection, including the steps of storing an injection force and loading an injectable into the nozzle of the needle-free injector. Some users may not be able to perform these preparation steps adequately due to reasons such as not having the required strength, or unsure of the amount of injectable to be loaded. A more reliable system is desired.

### SUMMARY OF INVENTION

In the light of the foregoing background, it is an object of the present invention to provide an alternate needle-free injector system.

Accordingly, the present invention, in one aspect, is a system comprising a needle-free injector having a needle-free injector comprising a trigger assembly adapted to exert an injection force, the trigger assembly comprising a spring storing the injection force. The system also comprises a winding mechanism for priming the spring for the storing of the injection force, wherein the winding mechanism is electrically powered.

In an exemplary embodiment of the present invention, the winding mechanism comprises an electric motor. The electric motor induces rotational movement of a first portion of the needle-free injector relative to a second portion of the needle-free injector. The rotational movement causes the priming of the spring.

In one embodiment, the system further comprises a locking mechanism for locking the first portion of the needle-free injector to the winding mechanism. At a locked state, the first portion of the needle-free injector is rotatable relative to the second portion for the priming of the spring.

In one embodiment, the first portion is axially movable relative to the locking mechanism while radially locked to the locking mechanism at the locked state.

According to another aspect of the present invention, an alternate needle-free injector system is disclosed. The system comprises a needle-free injector comprising a nozzle adapted to be loaded with an injectable. The system also comprises a dosing mechanism for loading the injectable into the nozzle, wherein the dosing mechanism is electrically powered.

In one embodiment, the dosing mechanism comprises an electric motor. The electric motor induces rotational movement of a first portion of the needle-free injector relative to the nozzle. The rotational movement causes the loading of the injectable into the nozzle.

In an exemplary embodiment, the electric motor is programmable to perform a predetermined number of rotations, thereby loading a predetermined dosage of the injectable into the nozzle.

In an exemplary embodiment, the system comprises a computing module for monitoring an amount of injectable loadable into the nozzle based on the predetermined dosage.

There are many advantages to the present invention. A main advantage is that the user no longer needs to prime the spring himself for injection. As the force required to prime the spring is significant, having an electrically powered winding mechanism ensures that every single user can appropriately prepare the injector for injection.

Another advantage of the present invention is that the dosage quantity of the injectable is stored in the base unit's memory, simplifying the dosing process and reducing the possibility of mistakes when dosing. It is especially useful when users may not be able to remember the correct dosage occasionally. The computing module and the warning mechanism further reduce the chance that the required dosage is not loaded due to having not enough injectable in the vial.

### BRIEF DESCRIPTION OF FIGURES

FIG. 1 is a perspective view of a needle-free injector system according to an embodiment of the present invention.

FIG. 2 is a cross-sectional view of the needle-free injector in FIG. 1 with the main spring at a primed state.

FIG. 3 is a perspective view of the winding mechanism according to an embodiment of the present invention.

FIG. 4 is a perspective view of the system in FIG. 1 with the case opened and the needle-free injector disposed within the base unit.

FIG. 5 is a cross-sectional view of a release mechanism according to an embodiment of the present invention.

### DETAILED DESCRIPTION OF THE PREFERRED EMBODIMENTS

As used herein and in the claims, “comprising” means including the following elements but not excluding others.

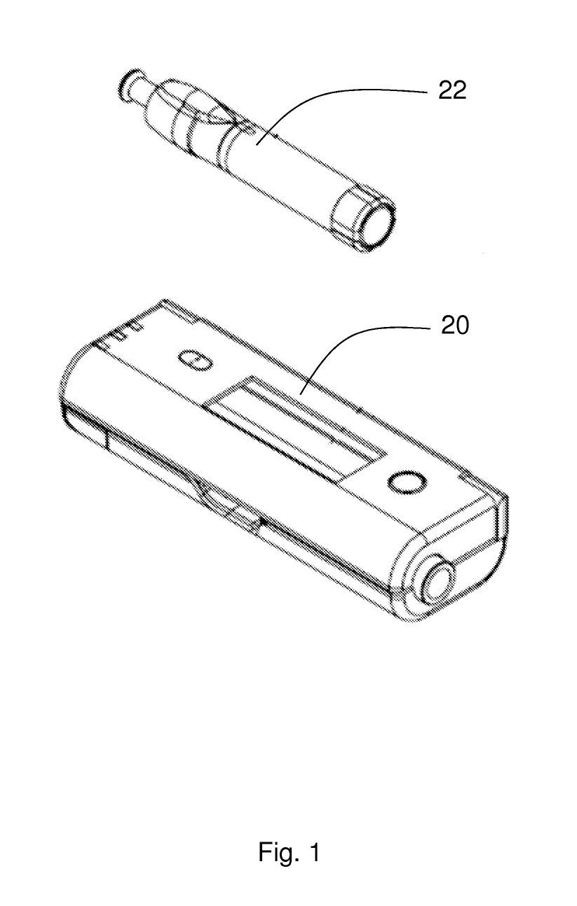

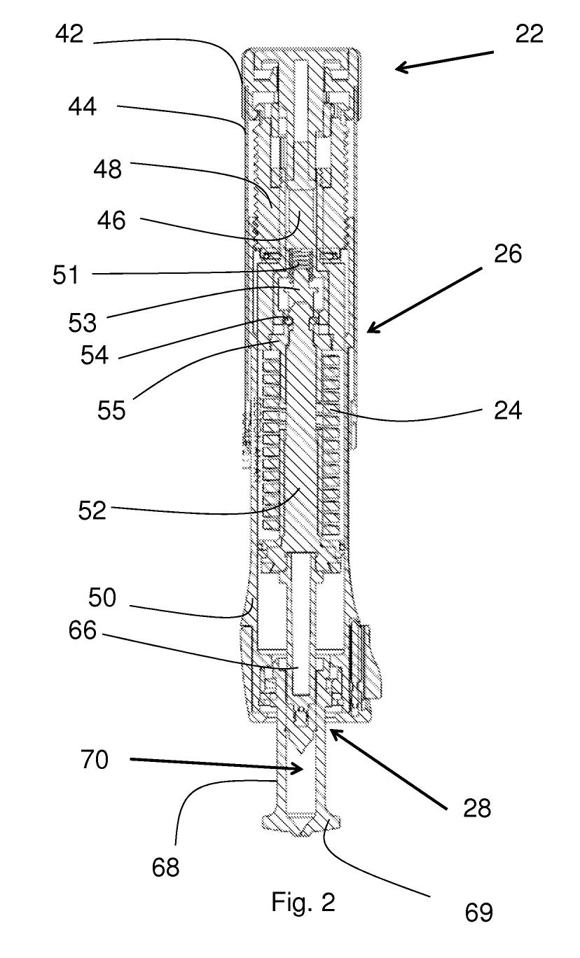

Referring now to FIGS. 1 and 2, the first embodiment of the present invention is an electrical needle-free injector system, comprising a base unit 20 and a needle-free injector 22. The needle-free injector 22 is adapted to be placed at a predetermined position within the base unit 20. The needle-free injector 22 comprises a main spring 24 that is adapted to be primed to store an injection force, an injector locking mechanism 26 to lock the main spring 24 in a primed state, and a nozzle 28 that is adapted to be loaded with an injectable and adapted to discharge the injectable upon exertion of the injection force stored in the main spring 24.

In a specific embodiment, the injector locking mechanism 26 comprises a sleeve 55 having a flange extending radially from the mid portion. The flange acts as the rear stop point of the main spring 24. The sleeve 55 allows a center shaft 52 to protrude there through. A plurality of openings are provided towards the rear end of the wall of the sleeve 55, with a ball bearing 54 disposed inside each opening and able to move radially inwards or outwards through the opening. A sleeve cover 53 having a flange extending radially outwards is provided rearwards of the sleeve 55 with the flange engaging with the rear edge of the wall of the sleeve 55. The rear end of the sleeve cover 53 is formed into a rounded bottom. A trigger spring 51 is provided with its front end engaging the sleeve cover 53 and adapted to urge the sleeve cover 53 towards the sleeve 55. The round bottom of the sleeve cover 53 acts as a trigger spring guide.

A retention member 46 is adapted to have a cavity opened from the front end thereof. This cavity is formed from two cylindrical spaces joined end-to-end, the first cylindrical space disposed at the front end having a larger diameter, and the second 100 cylindrical space with a smaller diameter extending towards the back end of the retention member 46. The rear end of the trigger spring 51 engages the retention member 46 by extending along the second cylindrical space. The front end of the first cylindrical space has a constricted section forming a neck, the neck at this initial state is in front of the ball bearings 54.

An exemplary embodiment with a more detailed description of the injector locking mechanism 26 can be found in the commonly owned U.S. application Ser. No. 12/973,890 filed on 20 Dec. 2010, which is hereby incorporated by reference in its entirety.

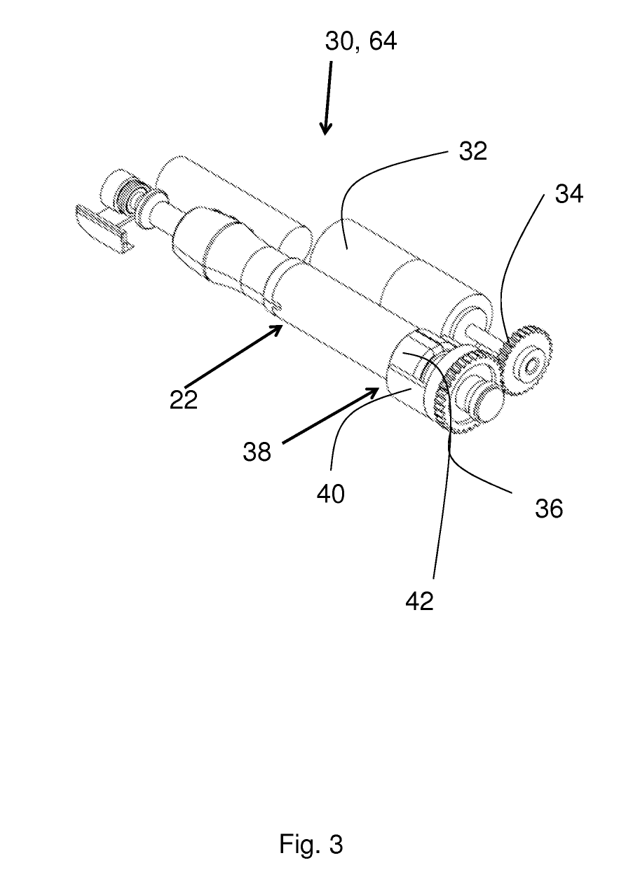

In an exemplary embodiment, the base unit 20 comprises an electrically powered winding mechanism 30 to prime the spring 24 of the needle-free injector electrically. In one embodiment as shown in FIG. 3, the winding mechanism comprises an electric DC motor 32 and a gear assembly 34 attached to the DC motor 32. The gear assembly 34 is coupled to a winding gear 36 that is attached to a locking mechanism 38 adapted to lock the needle-free injector at a predetermined position.

In an exemplary embodiment, the locking mechanism 38 comprises a holding member 40 adapted to engage with and lock to a first portion of the needle-free injector 22, and also adapted to attach to the winding gear 36. For the purposes of describing the winding mechanism as shown in FIG. 2, the first portion comprises a locking member 42, a back body 44 fixed to the locking member 42 and a cylinder bush 48 radially locked to the back body 44. In a specific embodiment, the holding member 40 of the base unit 20 is elastically deformable with a plurality of protrusions to engage with the locking member 42 of the needle-free injector 22.

In an operation of the winding mechanism 30, the DC motor 32 rotates, inducing or driving the winding gear 36 to rotate in a predetermined direction via the gear assembly 34, e.g. a clockwise direction. The locking member 40, being attached to the winding gear 36, also rotates as a result. While the locking member 40 rotates, the first portion of the needle-free injector 22 rotates relative to a second portion of the needle-free injector 22, the second portion comprising a front body 50 and the nozzle 28 in a specific embodiment as shown in FIG. 2 for illustration. A screw thread on the cylinder bushing 48 translates the rotational motion of the first portion into an axial motion, pushing the first portion towards the second portion. Components of the injector locking mechanism 26 other than the center shaft 52 are also pushed towards both the second portion and the center shaft 52. The main spring 24 reaches the primed state and is locked thereat when a locking groove of the center shaft 52 is level to the ball bearings 54, where the ball bearings 54 move radially inwards to engage the locking groove while the neck of the retention member 46 prevents the ball bearings 54 from moving radially outwards.

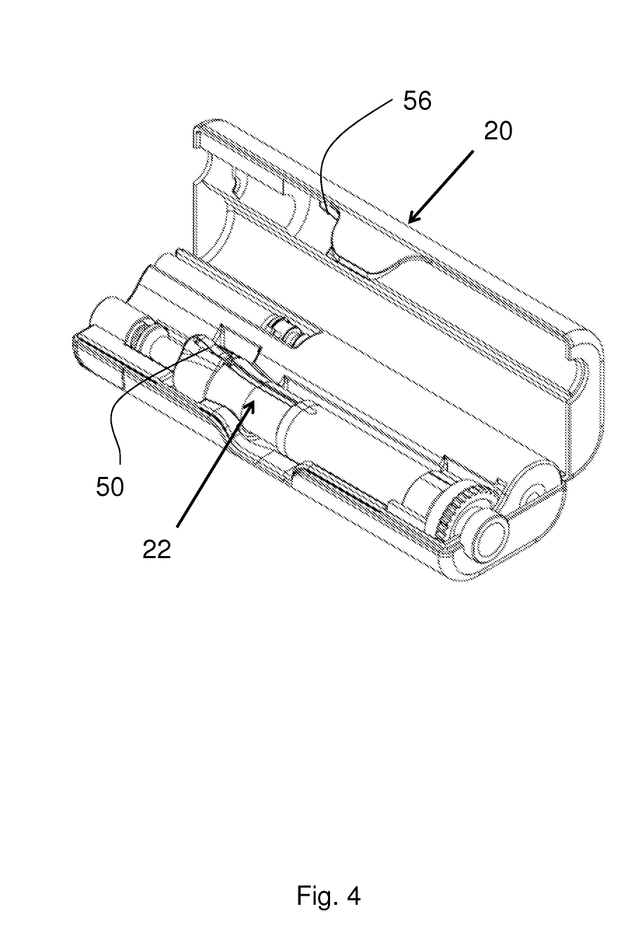

In one embodiment, the locking mechanism 38 comprises a blocking member 56 adapted to block the second portion from rotating, such that the first portion is allowed to rotate relative to the second portion. In an exemplary embodiment as shown in FIG. 4, the blocking member 56 is provided as a protrusion at an openable top part of a casing of the base unit 20. The second portion of the needle-free injector 22, for example at the front body 50, has a corresponding protrusion (not shown) adapted to engage with the blocking member 56.

In operation, firstly the top part of the casing is closed with the needle-free injector 22 disposed inside the casing. When winding starts, initially the first portion and the second portion both rotate in the predetermined direction. Along the rotation of the second portion, when the front body 50 is at a predetermined position, the blocking member 56 will engage with the protrusion of the front body 50, blocking the front body 50 and the second portion from further moving in the predetermined direction. The first portion continues to rotate and will result in a rotation relative to the second portion.

This configuration ensures that the winding mechanism 30 is operable without confirming the relative radial position of the second portion relative to the first portion before winding every time. A user then only needs to lock the first portion to the holding member 40 without the need of simultaneously being aware of the radial position of the second portion. Also, this configuration ensures that the winding mechanism 30 is only operable when the casing is closed.

In an exemplary embodiment, the locking mechanism 38 allows an axial motion of the first portion of the needle-free injector 22 while radially holding the first portion. This is because the total length of the needle-free injector 22 changes during the winding process, as the first portion including the rear end moves axially towards the second portion during the winding process. The locking mechanism 38 allowing the axial motion implies that the second portion is axially fixed. In an exemplary embodiment, the holding member 40 is elastically deformable with a plurality of protrusions adapted to engage with corresponding axial grooves of the locking member 42, the protrusions being able to axially slide in the axial grooves.

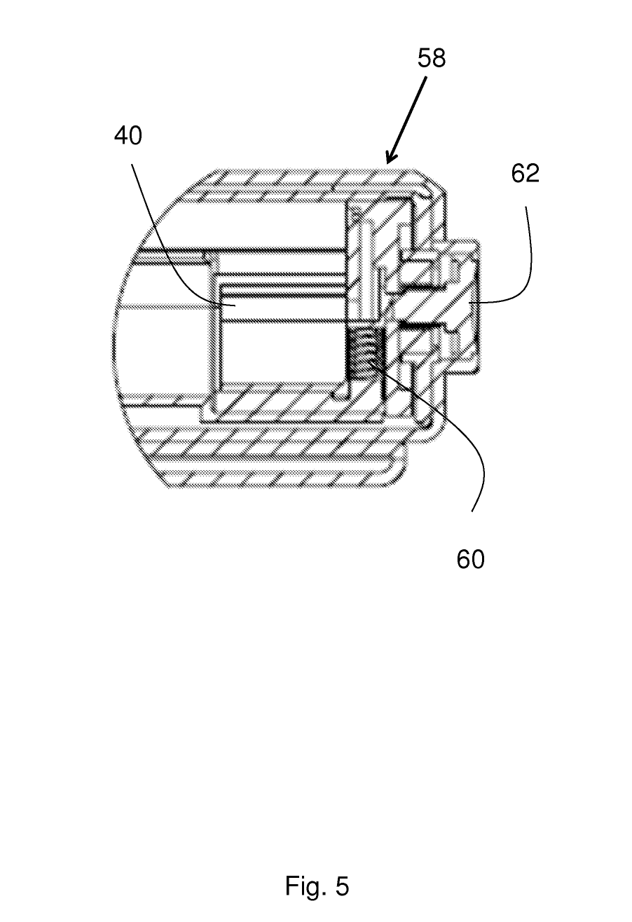

In an exemplary embodiment, the locking mechanism 38 further comprises a release mechanism 58 for releasing the needle-free injector 22 from the locked state. In one embodiment as shown in FIG. 5, the release mechanism 58 comprises a release spring 60 attached to the bottom end of the holding member 40, and comprises a release button 62 at the exterior of the base unit 20. When the user presses the release button 62, the release spring 60 exerts a release force to push the holding member 40 and the needle-free injector 22 upwards. The protrusions of the holding member 40 moves outwards at the same time, releasing the needle-free injector 22 from the locked state.

In an exemplary embodiment, the DC motor 32 of the winding mechanism 30 is programmable to perform a predetermined number of rotations. In one embodiment, the predetermined number of rotations is the number of rotations needed for priming the main spring 24.

In an exemplary embodiment, the base unit 20 further comprises a battery for powering the winding mechanism 30. In an exemplary embodiment, the battery is a rechargeable battery such as a lithium battery. An adaptor is provided to connect the base unit 20 to a power supply for recharging the battery.

In another aspect of the invention, the electrical needle-free injector system comprises an electrically powered dosing mechanism. In an exemplary embodiment, the system is similar to the system with the electrically powered winding mechanism as shown in FIGS. 1 and 2, comprising the base unit 20 and the needle-free injector 22, the needle-free injector 22 comprising the spring 24, the injector locking mechanism 26 and the nozzle 28. The base unit 20 comprises the electrically powered dosing mechanism 64, which in an exemplary embodiment shares the same components as the electrically powered winding mechanism 30 as shown in FIG. 3, such as the DC motor 32, the gear assembly 34, the winding gear 36 and the locking assembly 38.

In an operation of the dosing mechanism, it is first assumed that the spring 24 of the needle-free injector 22 is locked at the primed state, and a vial storing an injectable to be loaded is disposed at an appropriate position, e.g. an opening of the vial engaging to a head of the nozzle 28. The DC motor 32 rotates, preferably in an opposite direction to the operation of the winding mechanism, e.g. a counterclockwise direction. Similar to the above, the holding member 40 also rotates in the counterclockwise direction, causing the first portion of the needle-free injector to rotate relative to the front body 50. As the main spring 24 of the needle-free injector 22 is locked at the primed state, the main spring 24 with the injector locking mechanism 26 including the center shaft 52 will move away from the front body 50.

Referring back to FIG. 2, the nozzle 28 comprises a nozzle ram 66 telescopically inserted into a nozzle body 68, together defining an injectable chamber 70, with the nozzle ram 66 defining the rear surface and the nozzle body 68 defining the rest of the surfaces. The nozzle ram 66 is locked to the center shaft 52 while the nozzle body is locked to the front body 50. As such, when the center shaft 52 moves away from the front body 50, the nozzle ram 66 also moves away from the nozzle body 68, enlarging the injectable chamber 70 in the process. The injectable stored in the vial is then loaded into the injectable chamber 70 through a nozzle head 69 due to pressure difference.

In an exemplary embodiment, the DC motor 32 is programmable to perform a predetermined number of rotations. As the displacement of the nozzle ram 66 is proportional to the number of rotations, by specifying the number of rotations, a desired volume of injectable can be loaded into the nozzle 28. In one embodiment, the predetermined number of rotations is always in terms of full rotations.

In an exemplary embodiment, the dosing mechanism 64 further comprises a computing module for monitoring an amount of injectable loadable into the nozzle. In a specific embodiment, the amount of injectable loadable is calculated based on a default value and the required dosage. For example, the default value is a volume of a standard vial e.g. 3 ml. When a 1.2 ml of injectable is loaded into the nozzle 28, the loadable amount as calculated by the computing module decreases from 3 ml to 1.8 ml. In one embodiment, a reset button is provided to reset the loadable amount to the default value (e.g. 3 ml in the above example), for instance when the vial is replaced by a new one.

In an exemplary embodiment, the dosing mechanism 64 further comprises a warning mechanism. When the loadable amount is determined to be less than the required dosage as calculated from the predetermined number of rotations, an alert is provided to the user and the DC motor 32 is stopped from rotating. The alert can be provided as a video alert such as a flash, an audio alert such as a beep sound or other kinds of alerts. Following the above example, after two 1.2 ml doses are loaded, the loadable amount determined by the computing module should be 0.6 ml. If a further 1.2 ml dose is to be loaded, the computing module recognizes that there is not enough injectable left in the vial, and as such activates the warning mechanism to prompt the user to replace the vial and press the reset button before continuing the process.

In an exemplary embodiment, the winding mechanism 30 and the dosing mechanism 64 are provided in a single system. Upon receiving user's instruction to start the system, the winding mechanism 30 and the dosing mechanism 64 will operate in sequence. For example, the DC motor 32 will first rotate in a first direction e.g. clockwise to wind the main spring 24 until it reaches the primed state and locked thereat. Then the DC motor 32 automatically reverses the direction of rotation to load the predetermined dosage of injectable into the nozzle 28. The needle-free injector 22 therefore is prepared for injection in a single instruction.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

What is claimed is:

1. A system comprising:
a) a needle-free injector comprising a trigger assembly adapted to exert an injection force, said trigger assembly comprising a spring storing said injection force;
b) a winding mechanism for priming said spring for said storing of said injection force;
wherein said winding mechanism is electrically powered.

2. The system according to claim 1, wherein said winding mechanism comprises an electric motor, said electric motor induces rotational movement of a first portion of said needle-free injector relative to a second portion of said needle-free injector, said rotational movement causes said priming of said spring.

3. The system according to claim 2, wherein said electric motor is programmable to do a predetermined number of rotations.

4. The system according to claim 2, further comprising a locking mechanism for locking said first portion of said needle-free injector to said winding mechanism, wherein said first portion of said needle-free injector is rotatable relative to said second portion at a locked state for said priming of said spring.

5. The system according to claim 4, wherein said locking mechanism comprises a holding member adapted to engage with and lock to said first portion, said holding member is fixed to said winding mechanism and rotates during said priming of said spring to induce rotational movement of said first portion relative to said second portion.

6. The system according to claim 4, wherein said locking mechanism comprises a blocking member for blocking said second portion of said needle-free injector from rotating past a predetermined position, allowing said first portion of said needle-free injector to rotate relative to said second portion.

7. The system according to claim 4, wherein said first portion is axially movable relative to said locking mechanism while radially locked to said locking mechanism at said locked state.

8. The system according to claim 4, further comprising a release mechanism to mechanically disengage said locking mechanism from said first portion at said locked state.

9. The system according to claim 8, wherein said release mechanism comprises a release spring disposed below an elastically deformable member adapted to engage with and lock to said first portion, wherein said release spring exerts a release force to urge said first portion to disengage with said elastically deformable member upon activation of said release mechanism.

10. A system comprising:
a) a needle-free injector comprising a nozzle adapted to be loaded with an injectable;
b) a dosing mechanism for loading said injectable into said nozzle;
wherein said dosing mechanism is electrically powered.

11. The system according to claim 10, wherein said dosing mechanism comprises an electric motor, said electric motor induces rotational movement of a first portion of said needle-free injector relative to said nozzle, said rotational movement causing said loading of said injectable into said nozzle.

12. The system according to claim 11, wherein said electric motor is programmable to perform a predetermined number of rotations, thereby loading a predetermined dosage of said injectable into said nozzle.

13. The system according to claim 12, wherein said system comprises a computing module for monitoring an amount of injectable loadable into said nozzle based on the predetermined dosage.

14. The system according to claim 13, wherein said system further comprises a reset button for resetting said amount of injectable loadable to a default amount.

15. The system according to claim 13, wherein said system further comprises a warning mechanism for providing an alert to the user when said amount of injectable loadable is less than said predetermined dose.

16. The system according to claim 11, wherein said rotational movement of said first portion relative to said nozzle causes axial movement of a nozzle ram relative to a nozzle body, thereby enlarging an injectable chamber defined by said nozzle ram and said nozzle body, said enlarging causes said injectable to be loaded into said injectable chamber through a nozzle head.
